SEQUENCE LISTING (1 sequence)

SEQ ID NO: 1 - protein, 219 aa, Saccharomyces cerevisiae
LVPELNEKDDDQVQKALASRENTQLMNRDNIEITVRDFKTLAPRRWLNDTIIEFFMKYIEKSTPNTVAFNSFFYTNLSERGYQGVRRWMKRKKTQIDKLDKIFTPINLNQSHWALGIIDLKKKTIGYVDSLSNGPNAMSFAILTDLQKYVMEESKHTIGEDFDLIHLDCPQQPNGYDSGIYVCMNTLYGSADAPLDFDYKDAIRMRRFIAHLILTDALK

(19) United States
(12) Patent Application Publication
(10) Pub. No.: US 2013/0017554 A1
(43) Pub. Date: Jan. 17, 2013

(54) SUMO-SPECIFIC AFFINITY TAG

(71) Applicants: Oliver Kerscher, Williamsburg, VA (US); Megan Donaher, Chesterfield, VA (US); Zachary C. Elmore, Nashville, TN (US)

(72) Inventors: Oliver Kerscher, Williamsburg, VA (US); Megan Donaher, Chesterfield, VA (US); Zachary C. Elmore, Nashville, TN (US)

(73) Assignee: COLLEGE OF WILLIAM AND MARY, Williamsburg, VA (US)

(21) Appl. No.: 13/530,214

(22) Filed: Jun. 22, 2012

Related U.S. Application Data
(60) Provisional application No. 61/500,434, filed on Jun. 23, 2011.

(51) Int. Cl. C07K 14/395; C07K 19/00; G01N 33/566; G01N 21/64; C12N 15/31; C12N 15/63

(52) U.S. Cl. 435/7.1; 536/23.74; 435/320.1; 436/501; 530/350

(57) ABSTRACT

A new SUMO-specific affinity tag is described herein, based on the amino acid sequence from 403-621 of the SUMO protease Ulp1, and containing a crucial substitution of serine for cysteine. This affinity tag is particularly useful for a range of applications, including detection and affinity purification of sumoylated proteins from cell extracts.

### STATEMENT REGARDING FEDERALLY SPONSORED RESEARCH AND DEVELOPMENT

This invention was made with government support under Grant No. R15-GM085792, awarded by the National Institutes of Health. The government has certain rights in the invention.

### CROSS-REFERENCE TO RELATED APPLICATIONS

Not applicable.

### FIELD OF INVENTION

The field of the invention relates to a specific affinity tag for SUMO, as well as applications for use of the affinity tag, including purification and detection.

### BACKGROUND OF THE INVENTION

Ubiquitin and SUMO belong to a conserved family of post-translational modifiers that become covalently attached to specific proteins in a reversible manner (Kerscher et al. (2006) Annual Review of Cell and Developmental Biology 22, 159-180). Ubiquitin is best known for its role in the targeted destruction of proteins including key cell-cycle regulators, but also holds many non-proteolytic functions (Chen et al. (2009) Molecular Cell 33, 275-286). Sumoylation, on the other hand, does not directly target proteins for proteasomal degradation. Rather, modification of proteins with SUMO has been shown to modulate various cellular processes, including cell-cycle regulation, transcriptional activation, nucleocytoplasmic transport, DNA replication and repair, chromosome dynamics, apoptosis, ribosome biogenesis, and formation of nuclear bodies (Wang et al. (2009) Journal of Cell Science 122, 4249-4252). These functional distinctions between ubiquitin and SUMO have been further blurred by the recent discovery of SUMO-targeted Ubiquitin Ligases (STUbLs) that enable SUMO to play an indirect role in proteasome-mediated degradation (Perry et al. (2008) Trends in Biochemical Sciences 33, 201-208).

SUMO proteins are highly conserved from yeast to humans. Yeast cells express one SUMO protein, Smt3, while vertebrates express three isoforms, SUMO1, SUMO2, and SUMO3 (Wilkinson et al. (2010) Biochemical Journal 428, 133-145). SUMO2, SUMO3, and yeast Smt3 can form SUMO chains. SUMO1, on the other hand, lacks the internal lysine required for polymerization and may function as a chain terminator for SUMO2 and 3 chains (Matic et al. (2008) Mol Cell Proteomics 7, 132-144). All SUMO variants are conjugated to lysine residues of specific proteins, but only a fraction of these target proteins are modified with SUMO at any given time, as reported by Hannich et al. ((2005) Journal of Biological Chemistry 280, 4102-4110) and Wykoff et al. ((2005) Mol Cell Proteomics 4, 73-83).

In the budding yeast Saccharomyces cerevisiae, the ligation of SUMO to specific substrate proteins requires an E1 heterodimer (Aos1 and Uba2) that activates SUMO, as well as E2 (Ubc9) and E3 (Siz1, Siz2, and Mms21) enzymes that aid in the conjugation and ligation of SUMO to proper target proteins. Two yeast SUMO proteases, Ulp1 and Ulp2, contain a conserved cysteine protease domain that can remove the SUMO moiety from modified proteins. Recent evidence suggests that Ulp2, and its mammalian orthologs Susp1/SENP6 and SENP7 play a role in the removal of SUMO and SUMO chains from nuclear proteins, as reported by Baldwin et al. ((2009) Cell Cycle 8, 3406-3419), Bylebyl et al. ((2003) Journal of Biological Chemistry 278, 44113-44120), Kroetz et al. ((2009) Molecular Biology of the Cell 20, 2196-2206), Mukhopadhyay et al. ((2006) Journal of Cell Biology 174, 939-949), and Uzunova et al. ((2007) Journal of Biological Chemistry 282, 34167-34175). Ulp1, on the other hand, has two contrasting cellular functions. Ulp1 facilitates sumoylation by processing precursor SUMO into its conjugation competent form. Conversely, Ulp1 also facilitates desumoylation by removing SUMO from nuclear and cytosolic proteins after conjugation (Li et al. (1999) Nature 398, 246-251). Therefore, impairment of Ulp1 results in the accumulation of SUMO conjugates and the inability to carry out de novo sumoylation.

Ulp1 and several other SUMO proteases play important roles in mitosis. In budding yeast, Li et al. ((1999) Nature 398, 246-251) report that loss of Ulp1-mediated desumoylation leads to cell cycle progression defects and cell death. This observation suggests that Ulp1 plays a key role in the sumoylation dynamics of important cell cycle regulatory proteins.

One set of cytosolic substrates of the Ulp1 SUMO protease are the septins, as reported by Makhnevych et al. ((2007) Journal of Cell Biology 177, 39-49) and Takahashi et al. ((2000) Journal of Biochemistry 128, 723-725). The septins comprise an evolutionarily conserved class of GTPases that are implicated in bud-site selection, bud emergence and growth, microtubule capture, and spindle positioning (Spiliotis, E. T. (2010) Cytoskeleton 67, 339-345). The septins Cdc3, Cdc11, and Shs1 are subject to sumoylation.

It would be useful to identify features of Ulp1 required for substrate-targeting in vivo and in vitro, and to identify and analyze distinct mutations in Ulp1 that affect the targeting and retention to sumoylated target proteins at the bud-neck of dividing cells.

In particular, it would be useful to provide Ulpl fragments that bind tightly to SUMO and can be used for a range of applications including, for example, purification and detection of sumoylated proteins.

### BRIEF SUMMARY OF THE INVENTION

A new SUMO-specific affinity tag is described herein. This affinity tag is based on the amino acid sequence from 403-621 of the SUMO protease Ulp1 and requires a protease-inactivating mutation C580S. This affinity tag, referred to herein as “Ulp1(3)^{(C580S)}” (or simply “U-tag”), interacts strongly with SUMO, SUMO chains, and sumoylated proteins. Ulp1(3)^{(C580S)} is particularly useful for a range of applications including but not limited to: affinity purification of sumoylated proteins from cell extracts using an immobilized Ulp1(3)^{(C580S)} or matrix-bound Ulp1(3)^{(C580S)}, dimerization of SUMO-tagged proteins with Ulp1(³)^{(C580S)} fusion proteins, in vitro detection of sumoylated proteins using a fluorescently labeled Ulp1(3)^{(C580S)}, and association of purified SUMO chains to a Ulp1(3)^{(C580S)} fusion protein for downstream applications including in vitro ubiquitylation assays with SUMO-targeted Ubiquitin ligases.

Functionally equivalent versions of the Ulp1(3)^{(C580S)} protein are also contemplated, including truncated and lengthened versions, provided said functionally equivalent versions (1) contain the C580S mutant, and (2) have at least 80% amino acid homology with the Ulp1(3)^{(C580S)} polypeptide sequence as set forth in SEQ ID NO: 1. Amino acid residue 178 of SEQ ID NO: 1 comprises the crucial substitution of serine for cysteine.

In another aspect, a composition is described comprising DNA isolates encoding for Ulp1(3)^{(C580S)} proteins.

In another aspect, a composition is described comprising a recombinant expression vector including the DNA encoding for Ulp1(3)^{(C580S)} proteins.

It is an object of the present invention to provide a method for affinity purification of sumoylated proteins.

It is an object of the invention to provide a method for dimerization of SUMO-tagged proteins.

It is an object of the invention to provide a method for detection of sumoylated proteins in vitro.

It is an object of the invention to provide a method for detecting a SUMO-tagged protein comprising the sequential steps of: covalently bonding a fluorescent tag to the Ulp1(3)^{(C580S)} polypeptide to produce a labeled Ulp1(3)^{(C580S)} polypeptide, mixing a SUMO-tagged protein with said labeled Ulp1(3)^{(C580S)} polypeptide, and determining the extent of labeled Ulp1(3)^{(C580S)} polypeptide that is bound to SUMO-tagged proteins.

### BRIEF DESCRIPTION OF THE DRAWINGS

The summary above, and the following detailed description, will be better understood in view of the drawings that depict details of preferred embodiments.

FIG. 1A shows fluorescence microscopy images indicating the localization of GFP-tagged Ulp1 and Ulp1^{(C580S)} after nocodazole-induced G2/M arrest (YOK 1611 and YOK 1474). Note that only the Ulp1^{(C580S)} mutant can be visualized at the bud-neck of arrested cells. The arrowhead denotes the position of the bud-neck. FIG. 1B is an image of an SDS-PAGE gel confirming sumoylation of Cdc3

FIG. 2A shows fluorescence microscopy images indicating the localization of GFP-tagged Ulp1^{(C580S)} after G2/M arrest. The septin ring localization of Ulp1^{(C580S)} is indicated when present (arrowheads). FIG. 2B shows fluorescence microscopy images visualizing the localization of Smt3-GFP in G2/M-arrested wildtype (WT), ubc9-1, and siz1Δ siz2Δ strains (YOK 1857, YOK 2144, YOK 2143).

FIG. 3A shows a schematic representation of Ulp1 deletion and truncation mutants described herein, along with (at right) fluorescence microscopy images of G2/M-arrested cells expressing the GFP-tagged Ulp1 constructs that are schematically represented. FIG. 3B shows a schematic representation of Ulp1 deletion and truncation mutants described herein, along with (at right) fluorescence microscopy images of G2/M-arrested cells expressing the GFP-tagged Ulp1 constructs that are schematically represented. FIG. 3C is a graph quantifying the distinct subcellular localization of wildtype and mutant Ulp1 region 3 constructs.

FIG. 4 shows fluorescence microscopy images indicating the localization of GFP-tagged Ulp1 constructs in large-budded cells at 30° C. and 37° C., the non-permissive temperature for kap121-ts. The position of septin ring-localized Ulp1 constructs is indicated (arrowheads).

FIG. 5A and FIG. 5B are images from yeast two-hybid assays identifying Ulp1 domains required for interaction with SUMO.

FIGS. 6A-6D are SDS-PAGE images showing the ability of immobilized Ulp1(3)^{(C580S)} to bind SUMO and SUMO-modified proteins from yeast whole cell extracts.

FIG. 7A is an SDS-PAGE image showing that MBP-Ulp1(3)^{(C580S)} can serve as a SUMO2 binding platform for STUbL-mediated substrate ubiquitylation. FIG. 7B is a schematic model for using MBP-Ulp1(3)^{(C580S)} as a SUMO2 binding platform for substrate ubiquitylation

FIG. 8 shows an SDS-PAGE image of sumoylated proteins isolated from a complex mixture of yeast proteins after affinity purification using immobilized MBP-Ulp1(3)^{(C580S)} (i.e., MBP-Ulp1(3)^{(C580S)} bound to amylose resin). The left lane shows the whole cell extract, the center lane shows the proteins isolated using immobilized MBP-Ulp1(3)^{(C580S)}, and the right lane shows proteins isolated using amylose resin without bound MBP-Ulp1(3)^{(C580S)}.

FIG. 9 shows the amino acid sequence (SEQ ID NO: 1) for the Ulp1(3)^{(C580S)} polypeptide from the Ulp1 protein between residues 403 and 621, with the key mutation of C580S appearing in amino acid residue 178.

### DETAILED DESCRIPTION OF THE INVENTION

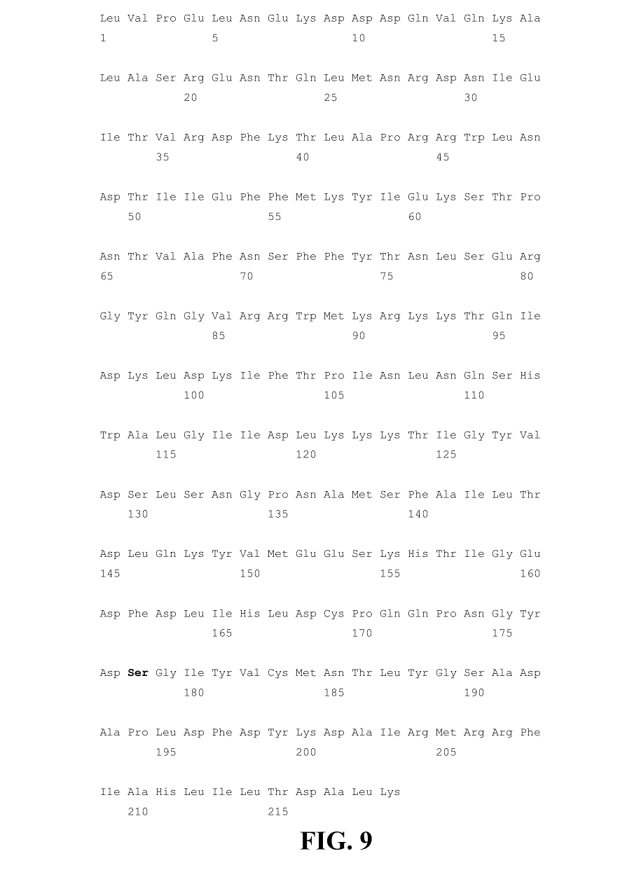

As used herein, abbreviations and names for proteins are consistent with terms generally used in the art. The term “Ulp1” refers to the Ubiquitin-Like Protein, systematic name YPL020C. SUMO (Small Ubiquitin-like Modifier) proteins are a family of small proteins that are covalently attached to and detached from other proteins in cells to modify their function. The term “C580S” means that the cysteine residue corresponding to position 580 in Ulp1 has been replaced with serine. In the 219 amino acid Ulp1(3)^{(C580S)} polypeptide described herein (see, for example, SEQ ID NO: 1 and FIG. 9), this substitution occurs at amino acid residue 178. The term “modification”, as used herein, covers substitution, insertion, deletion, or covalent addition of a protein onto a specific targeted protein. The terms “mutation” and “substitution” are used interchangeably herein. The term “variant” is intended to cover a polypeptide which differs in one or more amino acid residues from its parent polypeptide, typically in 1-20 residues.

A new SUMO-specific affinity tag is described herein. This affinity tag is based on the amino acid sequence from 403-621 of the SUMO protease Ulp1 and requires a protease-inactivating mutation C580S. This affinity tag interacts strongly with SUMO, SUMO chains, and sumoylated proteins, and is particularly useful for a range of applications including but not limited to: affinity purification of sumoylated proteins from cell extracts using an immobilized Ulp1(3)^{(C580S)} or matrix-bound Ulp1(3)^{(C580S)}, dimerization of SUMO-tagged proteins with Ulp1(3)^{(C580S)} fusion proteins, in vitro detection of sumoylated proteins using a fluorescently labeled Ulp1(3)^{(C580S)}, and association of purified SUMO chains to a Ulp1(3)^{(C580S)} fusion protein for downstream applications including in vitro ubiquitylation assays with SUMO-targeted Ubiquitin ligases.

Functionally equivalent versions of the Ulp1(3)^{(C580S)} protein are also contemplated, including truncated and lengthened versions, provided said functionally equivalent versions contain the C580S mutant and have at least 80% amino acid homology with the Ulp1(3)^{(C580S)} polypeptide sequence as set forth in SEQ ID NO: 1. Amino acid residue 178 of SEQ ID NO: 1 comprises the crucial substitution of serine for cysteine. In some embodiments, functionally equivalent versions of the Ulp1(3)^{(C580S)} protein have between 1 and 10 amino acid residue modifications relative to the Ulp1(3)^{(C580S)} polypeptide sequence as set forth in SEQ ID NO: 1.

In another aspect of the invention, a composition is described comprising DNA isolates encoding for Ulp1(3)^{(C580S)} proteins, or encoding for functionally equivalent versions of the Ulp1(3)^{(C580S)} protein. In another aspect, a composition is described comprising a recombinant expression vector including the DNA encoding for Ulp1(3)^{(C580S)} proteins, or for functionally equivalent variants of the Ulp1(3)^{(C580S)} protein.

Methods are described herein for affinity purification of sumoylated proteins, and also for detection of sumoylated proteins by binding them to fluorescently labeled Ulp1(3)^{(C580S)} protein, or functionally equivalent versions thereof. For example, one method for detecting a SUMO-tagged protein comprises the sequential steps of: covalently bonding a fluorescent tag to the Ulp1(3)^{(C580S)} polypeptide to produce a labeled Ulp1(3)^{(C580S)} polypeptide, mixing a SUMO-tagged protein with said labeled Ulp1(3)^{(C580S)} polypeptide, and determining the extent of labeled Ulp1(3)^{(C580S)} polypeptide that is bound to SUMO-tagged proteins.

The methods of the invention also contemplate dimerization of SUMO-tagged proteins.

Fusion protein comprising Ulp1(3)(C580S) can only be produced according to the methods of the invention. For example, a fusion protein can be synthesized comprising a Ulp1(3)(C580S) protein fused at the C terminus with a protein or peptide capable of being fused to Ulp1(3)(C580S). Alternatively, a fusion protein can be synthesized comprising a Ulp1(3)(C580S) protein fused at the N terminus with a protein or peptide capable of being fused to Ulp1(3)(C580S).

### EXAMPLES

Exemplary investigations supporting the compositions and methods of the present invention are presented below. The examples that follow are intended in no way to limit the scope of this invention, but are provided to illustrate representative embodiments of the present invention. Many other embodiments of this invention will be apparent to one skilled in the art.

Experimental Procedures.

Yeast strains, media and plasmids. Yeast strains and plasmids used in this study are listed in Table 1. Yeast media preparation and manipulation of yeast cells was performed as previously published (see Guthrie, C., and Fink, G. R. (1991) Guide to yeast genetics and molecular biology, Academic Press, San Diego). Yeast strains were grown at 30° C. unless otherwise indicated. DNA fragments containing Ulp1 under the control of its endogenous promoter were amplified from yeast genomic DNA and placed in-frame with a carboxy-terminal GFP tag in the CEN/LEU2 plasmid pAA3 (44). Primer pairs used for full-length Ulp1 amplification were OOK2 (ULP1 (−310 to −294)) and OOK3 (ULP1 (+1842 to +1863)). To prepare truncated and mutated Ulp1-GFP constructs listed in Table 1 below, Quikchange XL Site-Directed Mutagenesis (Stratagene) and Phusion Site-Directed Mutagenesis kits (Finnzyme) were used according to manufacturers' instruction. Primer sequence information for the construction of individual mutants and truncations are available upon request. All constructs were sequenced verified and/or confirmed in complementation assays. For two-hybrid constructs, ORFS of the indicated genes were PCR-amplified and recombined into gapped pOAD and pOBD2 vectors (Yeast Resource Center, WA). To overexpress and purify Ulp1 truncations from bacteria, the respective Ulp1 fragments were PCR-amplified and cloned into pMALc-HT (obtained as a gift from Sean Prigge, JHSOM), thereby adding an in-frame maltose-binding protein (MBP) module followed by a TEV protease cleavage site and a His⁶ epitope tag. Ulp1 derivatives were expressed as MBP fusions in BL21 Star (DE3) cells containing a pRIL plasmid.

TABLE 1
Yeast Strains and Plasmids

| | Pertinent Genotypes or Parent | | |
|---|---|---|---|
| Name | Strain | Plasmids | Reference |
| MHY500 | Matα his3-Δ200 leu2-3,112 | | Li and Hochstrasser, 2003 |
| | ura3-52 lys2-801trp1-1 | | |
| | gal2 | | |
| BY4743 | MATa leu2Δ0 met15Δ0 ura3Δ0 | | Winzeler et al., 1999 |
| YOK 1611 | MHY500 | ULP1-GFP/LEU2 | This study |
| YOK 1474 | ″ | ULP1^{(C580S)}-GFP/LEU2 | ″ |
| YOK 1490 | ″ | ULP1(Reg1)-GFP/LEU2 | ″ |
| YOK 1861 | ″ | UlLP1(Reg2)-GFP/LEU2 | ″ |
| YOK 1479 | ″ | ULP1(Δ2)-GFP/LEU2 | ″ |
| YOK 2016 | ″ | ULP1^{(D451N C580S)}-GFP/LEU2 | ″ |
| YOK 1839 | ″ | ULP1(Reg3)-GFP/LEU2 | ″ |
| YOK 1907 | ″ | ULP1(Reg3^{(C580S)})- | ″ |
| | | GFP/LEU2 | |
| YOK 1903 | ″ | ULP1((Reg3ΔSBS)- | ″ |
| | | GFP/LEU2 | |
| YOK 2203 | ″ | ULP1(SBS)-GFP/LEU2 | ″ |
| YOK 1828 | ″ | ULP1((Reg3^{(ts)})-GFP/LEU2 | ″ |
| YOK 2157 | ″ | ULP1((Reg3^{(ts C580S)})- | ″ |
| | | GFP/LEU2 | |
| YOK 1857 | ″ | SMT3-GFP/LEU2 | plasmid published in Panse |
| | | | et al., 2003 |
| YOK 44 | smt3-331 | | Biggins et al., 2001 |
| YOK 1995 | ″ | ULP1^{(C580S)}-GFP/LEU2 | This study |
| YOK 847 | ubc9-1 | | Betting and Seufert, 1996 |
| YOK 2065 | ″ | ULP1^{(C580S)}-GFP/URA3 | This study |
| YOK 2144 | ″ | SMT3-GFP/URA3 | ″ |
| GBY1 | MATa smt3 R11,15,19::TRP1 | | Bylebyl et al., 2003 |
| YOK 1910 | GBY1 | ULP1^{(C580S)}-GFP/LEU2 | This study |
| yDS880 | MATa-inc ade2-101 his3-200 | | Schwartz et al., 2007 |
| | leu2-1::GAL-HO-LEU2 lys2-801 | | |
| | RAD53::FLAG-HIS3 siz1::NAT | | |
| | siz2::HPH sml1::KAN trp1-63 | | |
| | ura3-52 VII-L::TRP-HO site- | | |
| | LYS2 | | |
| YOK 2067 | MATa-inc ade2-101 his3-200 | ULP1^{(C580S)}-GFP/URA3 | This study |
| | leu2-1::GAL-HO-LEU2 lys2-801 | | |
| | RAD53::FLAG-HIS3 siz1::NAT | | |
| | siz2::HPH sml1::KAN trp1-63 | | |
| | ura3-52 VII-L::TRP-HO site- | | |
| | LYS2 | | |
| YOK 2143 | MATa-inc ade2-101 his3-200 | SMT3-GFP/URA3 | ″ |
| | leu2-1::GAL-HO-LEU2 lys2-801 | | |
| | RAD53::FLAG-HIS3 siz1::NAT | | |
| | siz2::HPH sml1::KAN trp1-63 | | |
| | ura3-52 VII-L::TRP-HO site- | | |
| | LYS2 | | |
| kap121ts | kap121::ura3::HIS3 ura3-52 | pRS314-kap121-34 | Leslie et al., 2002 |
| | his3Δ200 trp1-1 leu2-3,112 | | |
| | lys-2-801 | | |
| YOK 1487 | kap121ts | ULP1-GFP/LEU2 | This study |
| YOK 1488 | kap121ts | ULP1^{(C580S)}-GFP/LEU2 | ″ |
| YOK 1944 | kap121ts | ULP1(Reg3^{(C580S)})- | ″ |
| | | GFP/LEU2 | |
| AH109 | MATa, trp1-901, leu2-3, 112, | | Clontech, CA |
| | ura3-52, his3-200, gal4Δ, | | Cat. # 630444 |
| | gal80Δ, LYS2::GAL1UAS- | | |
| | GAL1TATA-HIS3, GAL2UAS- | | |
| | GAL2TATA-ADE2, | | |
| | URA3::MEL1UASMEL1TATA- | | |
| | lacZ, MEL1 | | |
| YOK 2163 | AH109 | ULP1-pOAD/LEU2 + | This study |
| | | SMT3-pOBD/TRP1 | |
| YOK 2165 | ″ | ULP1^{(C580S)}-pOAD/LEU2 + | ″ |
| | | SMT3-pOBD/TRP1 | |
| YOK 2167 | ″ | ULP1 (Reg1)-pOAD/LEU2 + | ″ |
| | | SMT3-pOBD/TRP1 | |
| YOK 2169 | ″ | ULP1 (Reg2)-pOAD/LEU2 + | ″ |
| | | SMT3-pOBD/TRP1 | |
| YOK 2171 | ″ | ULP1 (Reg3)-pOAD/LEU2 + | ″ |
| | | SMT3-pOBD/TRP1 | |
| YOK 2173 | ″ | ULP1 (Reg3^{(C580S)})- | ″ |
| | | pOAD/LEU2 + SMT3- | |
| | | pOBD/TRP1 | |
| YOK 2175 | ″ | ULP1 (Reg3^{(D451N)})- | ″ |
| | | pOAD/LEU2 + SMT3- | |
| | | pOBD/TRP1 | |
| YOK 2177 | ″ | ULP1 (Reg3^{(ts)})- | ″ |
| | | pOAD/LEU2 + SMT3- | |
| | | pOBD/TRP1 | |
| YOK 2179 | ″ | ULP1 (Reg3^{(ts S450N)})- | ″ |
| | | pOAD/LEU2 + SMT3- | |
| | | pOBD/TRP1 | |
| YOK 2181 | ″ | ULP1 (Reg3^{(ts C580S)})- | ″ |
| | | pOAD/LEU2 + SMT3- | |
| | | pOBD/TRP1 | |
| YOK 2183 | ″ | SMT3-pOAD/LEU2 + SLX5 | ″ |
| | | pOBD/TRP1 | |
| YOK 2185 | ″ | vector-pOAD/LEU2 + | ″ |
| | | vector pOBD/TRP1 | |
| YOK 428 | ulp1::KAN (segregant of | ulp1ts/TRP/NAT | ″ |
| | heterozygous diploid | GPD-FLAG- | |
| | ULP1/ulp1::KAN in BY4743- | SMT3gg/pRS425 | |
| | (OpenBiosystems, Huntsville, | | |
| | AL. -- Cat.#YSC1021-671376) | | |

Yeast Two-Hybrid Assays- Gal4-activation-domain (AD) fusions of ULP1 and the indicated ULP1 mutants in pOAD were transformed into the AH109 reporter strain expressing a Gal4-DNA-binding-domain (BD) fusion of SMT3 in pOBD. Two-hybrid interactions of serially diluted cells were scored in duplicate on dropout media lacking adenine.

Pulldown Assays, Affinity Purification, and Protein extracts—Frozen bacterial cell pellets from 200 ml of IPTG-induced BL21 Star (DE3) cells were thawed on ice and re-suspended in 2 ml 1× phosphate buffered saline (PBS) containing 1× Halt Protease Inhibitor Cocktail (Pierce Cat. # 78430). Ice-cold cells were sonicated using a Branson Sonifier, and extracts were cleared by centrifugation at 15k RPM for 8 minutes at 4° C. Cleared bacterial extracts were added to 15 mL conical tubes and diluted using 4 mL 1× PBS containing the protease inhibitor cocktail. MBP-tagged proteins (MBP-Ulp1(3), Ulp1(3)^{(C580S)}, or Ulp1(3)^{(C580S)}ΔSBS) were bound to 5 ml columns containing 300 μl amylose resin (New England Biolabs) and washed extensively with 1× PBS. Yeast cell protein extracts containing the indicated target proteins were passed over the amylose resin ,and proteins bound to MBP-Ulp1(3), Ulp1(3)^{(C580S)}, or Ulp1(3)^{(C580S)}ΔSBS were eluted with 100 mM maltose or SDS-PAGE sample buffer. Yeast cell protein extracts were generated by bead-beating ˜50 ODs of yeast cell pellets in 1× Cell Lysis buffer (#9803—Cell Signaling Technology, MA) containing 25 mM N-Ethylmaleimide (NEM). For SUMO pulldown experiments, recombinant MBP-Ulp1(3)^{(C580S)} or MBP-Ulp1(3) was incubated with SUMO-1 or SUMO-2 agarose (Boston Biochem) in 1 ml of 1× PBS with protease inhibitors (Thermo Scientific). Proteins bound to the agarose beads were washed in 1× PBS and eluted with 1× SDS-PAGE sample buffer. All protein extracts were separated on NOVEX 4-12% BIS-TRIS gradient gels (Invitrogen #NP0321) using MOPS-SDS running buffer (Invitrogen #NP0001).

Fluorescent Microscopy—Unless otherwise noted, cells were grown in rich media, arrested in G2/M using nocodazole (15 μg/ml/3h/30° C.), washed in 2% dextrose, and harvested by centrifugation. Images of live cells were collected using a Zeiss Axioskop fitted with a Retiga SRV camera (Q-imaging), i-Vision software (BioVision Technologies), and a Uniblitz shutter assembly (Rochester, N.Y.). Pertinent filter sets for the above applications include CZ909 (GFP), XF114-2 (CFP), XF104-2 (YFP) (Chroma Technology Group). Images were normalized using i-Vision software and pseudo-colored and adjusted using Adobe Photoshop software (Adobe Systems Inc.).

In vitro ubiquitylation reactions, recombinant proteins, and antibodies—Enzymes and substrates used in our in vitro ubiquitylation assays were quantified using a Protein 230 kit on the Agilent 2100 Bioanalyzer according to the manufacturer's instructions. 10× ubiquitylation buffer, E1 enzyme (Uba1), ATP, and 20× ubiquitin were provided in a commercial ubiquitylation kit (Enzo # BML-UW0400). Ubiquitylation buffer, IPP (100 U/ml), DTT (50 μM), E1 (Uba1), E2 (Ubc4), and E3 enzymes (RNF4) were combined with purified SUMO2 chains (#ULC-210—Boston Biochem, MA) and ubiquitin. Reactions totaled 27 μL and were incubated at 30° C. for three hours. Reactions were stopped by adding an equal volume of SUTEB sample buffer (0.01% bromophenol blue, 10 mM EDTA, 1% SDS, 50 mM Tris at pH 6.8, 8 M Urea) containing DTT (5 μL of 1 M DTT/1 mL SUTEB sample buffer). Protein products were boiled in a 65° C. heat block for ten minutes and analyzed by Western blot with anti-human SUMO2 antibody. Antibody sourcing was a follows: anti-human SUMO2 # BML-PW0510-0025 (ENZO Life sciences, PA), anti-GFP: JL8 # 632380 (Clontech, CA), anti-FLAG(M2) #F3165 (Sigma-Aldrich, MO), anti-PGK: 22C5 # 459250 (Invitrogen, CA), anti Cdc11 (y415):sc-7170 (Santa Cruz Biotechnology, CA).

### Example 1

### Localization of Ulp1 and the Catalytically Inactive Ulp1^{(C580S)} in Dividing Yeast Cells

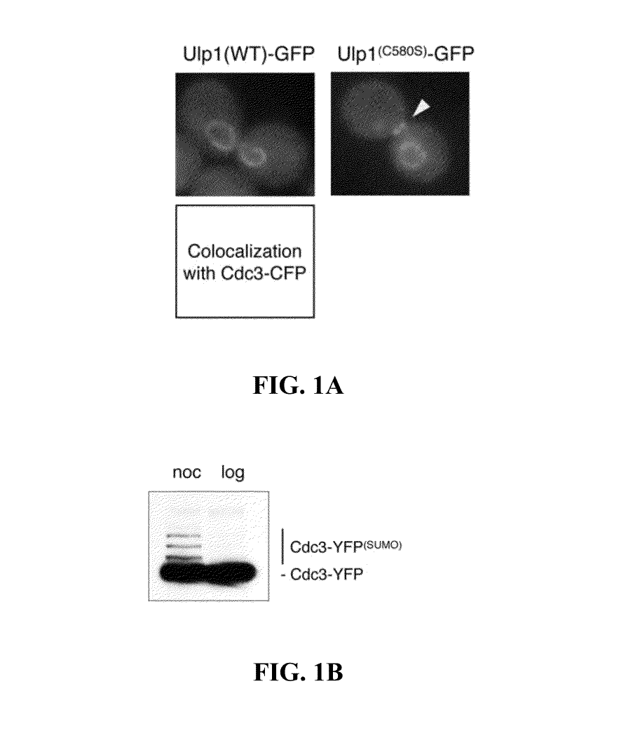

The localization of green-fluorescent protein (GFP)-tagged versions of both the full-length wildtype Ulp1 (WT) and a catalytically inactive mutant of Ulp1 (Ulp1^{(C580S)} in G2/M-arrested yeast cells was analyzed (see materials and methods). The C580S mutation replaces the catalytic cysteine with a serine residue, rendering the Ulp1 SUMO protease catalytically inactive. Both fusion proteins were expressed under the control of the Ulp1 promoter on low-copy plasmids, and images were collected using a fluorescent microscope. Shown in FIG. 1A are representative images indicating the localization of GFP-tagged Ulp1 and Ulp1^{(C580S)} after nocodazole-induced G2/M arrest (YOK 1611 and YOK 1474). Note that only the Ulp1^{(C580S)} mutant can be visualized at the bud-neck of arrested cells. The arrowhead denotes the position of the bud-neck. Unexpectedly, however, full-length Ulp1^{C580S} was enriched both at the bud-neck and the nuclear envelope of G2/M-arrested cells (FIG. 1A—right). This bud-neck localization of Ulp1^{C580S} is reminiscent of the localization of the septin ring.

Referring to FIG. 1B, whole cell extracts (WCEs) from yeast cells expressing the YFP-tagged septin Cdc3 (YOK 1398) were treated with nocodazole (noc) or grown logarithmically (log) prior to preparation of whole cell extracts. Extracted proteins were then separated on SDS-PAGE gels and probed with the JL-8 antibody (see materials and methods) to detect Cdc3-YFP and slower migrating sumoylated Cdc3-YFP adducts. Identity of sumoylated Cdc3-YFP bands was confirmed using gel-shift assays with FLAG-tagged Smt3 (data not shown). Several sumoylated septins have been shown to be Ulp1 substrates and herein it is demonstrated that the septin Cdc3 is highly sumoylated during G2/M arrest (FIG. 1B). Furthermore, a catalytically inactive Ulp1 mutant co-localizes with the septin Cdc11 in G2/M arrested (noc) cells. Therefore, Ulp1^{C580S} resides at the bud-neck localized septin-ring. This data suggests that introducing the C580S mutation into the catalytic domain of Ulp1 alters the sub-cellular distribution of this SUMO protease, causing it to localize with a bud-neck associated substrate, possibly a sumoylated septin protein.

### Example 2

### SUMO is Required for the Localization of Ulp1^{(C580S)} to the Septin Ring

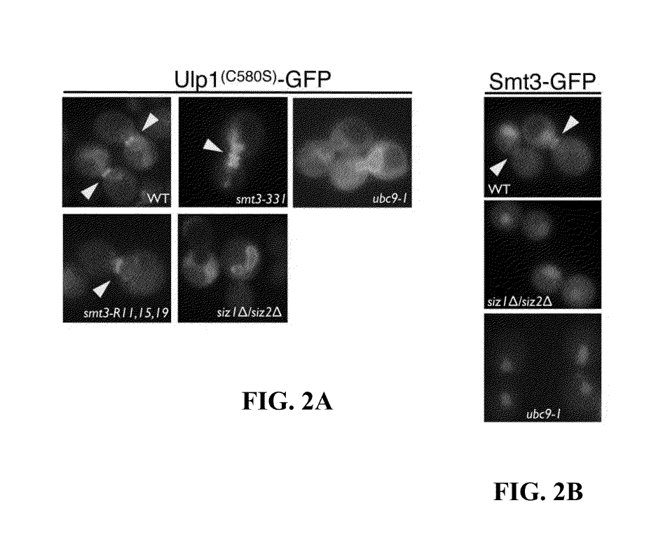

The next step was to determine whether the C580S mutation that visually increased the ability of Ulp1 to associate with the septin ring in vivo was, in fact, SUMO-dependent. For this purpose, the Ulp^{1C580S} construct was expressed in two Smt3 mutants (smt3-331 and smt3-R11,15,19) or two SUMO pathway mutants (ubc9-1, siz1Δ siz2Δ), along with a wildtype control strain (WT). Logarithmically growing cells of each mutant were arrested in G2/M, and images were collected to assess the septin ring localization of Ulp^{1C580S} in comparison to an SMT3 wildtype strain. In our analyses, we found that in both the absence of SUMO chains (in the R11,15,19 mutant) and improperly formed SUMO chains (in the smt3-331 mutant), the localization of Ulp^{1C580S} to the septin ring was reduced but not abolished in frequency and intensity (FIG. 2A). Shown are representative images indicating the localization of GFP-tagged Ulp^{1(C580S)} after G2/M arrest. The septin ring localization of Ulp^{1(C580S)} is indicated when present by the arrowheads in FIG. 2A. Note that ^{Ulp}^{1(C580S)} fails to localize to the septin ring in SUMO-conjugating and ligating enzyme mutants (ubc9-1 and siz1α siz2Δ, respectively). The ubc9-1 strain is a mutant of the SUMO E2 conjugating enzyme which impairs SUMO conjugation, and the siz1Δ siz2Δ strain is a SUMO E3 ligase double mutant that lacks sumoylation of septins and many other proteins. Consistent with a role for Smt3 in the localization of Ulp^{1C580S}, we were unable to detect septin ring localization of Ulp^{1C580S} in ubc9-1 and siz1Δ siz2Δ strains. However, Ulp^{1C580S} was retained at the nuclear envelope (FIG. 2A). As an additional control, the septin ring localization of GFP-tagged Smt3 was undetectable in both ubc9-1 and siz1Δ siz2Δ strains (FIG. 2B).

In summary, Smt3 is required for Ulp1 localization to the septin ring. Therefore, Ulp1 is targeted to the septin ring of dividing cells in a SUMO-dependent fashion. Our data also suggests that the formation of SUMO chains on substrates may enhance this targeting of Ulp1.

### Example 3

### Distinct and Separate Ulp1 Domains are Required for Localization to the Septin Ring

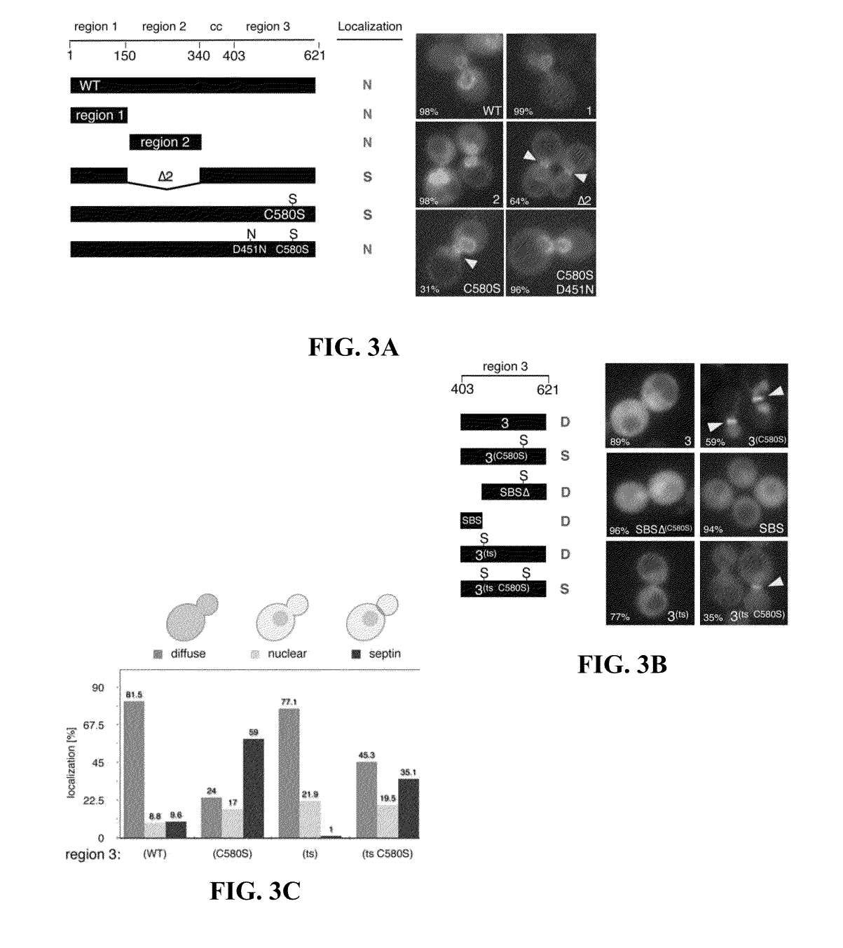

Our finding that a single point mutation in Ulp1, C580S, dramatically enhanced the localization of full-length Ulp1 to the septin ring in a SUMO-dependent fashion warranted a more detailed analysis of the targeting domains in Ulp1. Therefore, we generated a collection of GFP-tagged Ulp1 truncations and domains that were expressed under control of the Ulp1 promoter. We reasoned that the truncations and domains of Ulp1 that retained substrate targeting information would also localize to the septin ring in G2/M-arrested cells. In all, we assessed the localization of ten GFP-tagged constructs in comparison to full-length wildtype Ulp1 (WT) and full-length Ulp1^{C580S} (C580S). Our choice of individual constructs was guided by previous findings that Ulp1 consists of functionally separate domains. These domains include a Kap121-binding domain with a role in septin localization (region 1), a Kap95-Kap60-binding domain with a role in NPC anchoring (region 2), a coiled-coil domain harboring a nuclear export signal (CC), and the catalytic ubiquitin-like protease domain (UD) (region 3) (25-27). Depictions and images of these domains and their subcellular localizations are shown in FIGS. 3A and 3B. The length of each construct (amino acid scale: 1-621), individual domains of Ulp1, and pertinent amino acid changes are shown. WT: full-length Ulp1; region 1: Ulp1(1-150); region 2: Ulp1(151-340); region 3: Ulp1(341-621); Δ2: Ulp1 lacking region 2; C580S: catalytically inactivating mutation; D451 N: deleted salt-bridge with SUMO (YOK 1611, YOK 1474, YOK 1490, YOK 1861, YOK 1479, YOK 2016, YOK 1839, YOK 1907, YOK 1903, YOK 2203, YOK 1828, YOK 2157). The letters N, S, and D summarize the observed nuclear, septin or diffuse localization of the indicated constructs, respectively. SBS corresponds to a shallow SUMO-binding surface on Ulp1 (31,56,57). On the left side of FIGS. 3A and 3B are schematic representations of these Ulp1 deletion and truncation mutants used. On the right side of FIGS. 3A and 3B are representative images of G2/M-arrested cells expressing the GFP-tagged Ulp1 constructs shown on the left. The fraction of cells (%) with N, S, or D localization and the presence and position of septin ring-localized Ulp1 constructs is indicated (arrowheads). FIG. 3C is a graph quantifying distinct subcellular localization of wildtype and mutant Ulp1 region 3 constructs. Large-budded G2/M-arrested cells were imaged to assess either diffuse, nuclear, or septin ring localization (n>100).

We demonstrated that the Ulp1 protein lacking region 2, (Δ2) localized to the septin ring in the majority of large-budded, arrested cells (27). Therefore, region 2 of Ulp1 normally antagonizes localization and/or retention at the septin ring. This result is complemented by our novel finding that the full-length Ulp1^{C580S} localized to the septin ring in 33% of all arrested, large-budded cells (FIGS. 1A and 3A).

Aspartate 451 (D451) in Ulp1 is required to form an essential salt-bridge with arginine 64 of Smt3. Therefore, we introduced a D451 N mutation into Ulp1^{C580S} and found that it abolished the accumulation of the full-length Ulp1 double mutant (D451 N, C580S) at the septin ring (FIG. 3A). This finding underscores the importance of Smt3 in targeting full length Ulp1 to the septin ring shown in FIG. 2A. Additionally, it may indicate that aspartate 451 is required for targeting of sumoylated proteins while the C580S mutation is required for retention of Ulp1 at the septin ring.

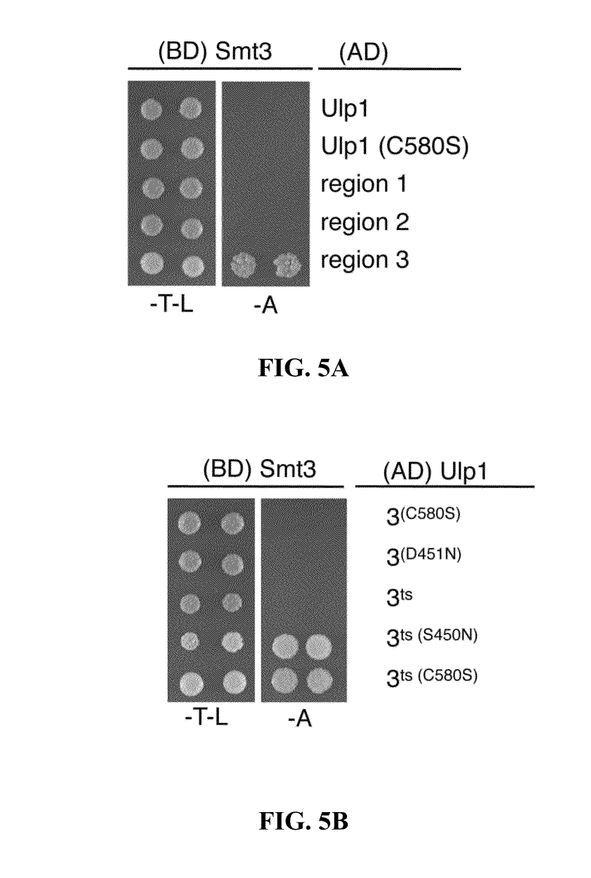

Most intriguingly, we found that a truncation consisting only of region 3 with the C580S mutation (Ulp1(3)^{(C580S)}) displayed robust septin ring localization in 59% of cells (FIG. 3B). In stark contrast, regions 1, 2, and wildtype region 3, lacking the C580S mutation, failed to localize to the septin ring (FIG. 3A and 3B). Therefore, necessary and sufficient SUMO-dependent targeting information is contained in region 3 of Ulp1 but not regions 1 and 2. The latter conclusion is also confirmed by two-hybrid assays with Smt3 (see FIG. 5A).

The previously published co-crystal structure of Ulp1 with Smt3 (MMDB database # 13315) reveals that amino acids 418-447 of region 3 make extensive contact with Smt3 and constitute an exposed SUMO-binding surface (“SBS”). In an attempt to identify critical residues in the evolutionary conserved SBS domain, we used psi-blast to compare the protein sequence of the yeast Ulp1 catalytic domain to all non-redundant protein sequences in the NCBI database for seven iterations and limited the output to the top 250 matches. Our results contained 81 different species; 61% of the sequences were identified as verified or predicted sentrin/SUMO protease/Ulp1 genes, 24% were identified as unnamed protein products or hypothetical genes and 15% were “other” (crystal structures, unanalyzed sequence, etc.). The alignment of these sequences allowed us to identify areas of strong conservation.

We also investigated the effect of deleting the entire SBS domain on the localization of Ulp1(3)^{(C580S)}. A Ulp1(3)^{(C580S)}SBSΔ construct does not localize to the septin ring in the majority of cells (96%). We also cloned and expressed the SBS domain as a fusion with GFP (SBS-GFP). This construct distributed diffusely throughout the cell and failed to localize to the septin ring. These data suggest that the SBS domain of region 3 may be required for the initial interaction with sumoylated substrates, but additional features of Ulp1 are required for targeting (D451) and retention (C580S) of this SUMO protease at the septin ring.

Next, we directed our attention to the conserved asparagine 450 (N450) residue that resides immediately next to the SBS domain. N450 is mutated in region 3 of the temperature-sensitive ulp1ts-333 allele which arrests in mitosis and accumulates unprocessed SUMO precursor and sumoylated proteins (Li et al. (1999) Nature 398, 246-251). Our ulp1ts construct of region 3, Ulp1(3)^{ts}, contains three mutations (I435V, N450S, I504T), and introduction of C580S into Ulp1(3)^{ts} greatly reduced the incident and intensity of septin ring localization (compare panels in FIGS. 3B and 3C). We noted that the (N450S) mutation in the is construct was located next to the salt-bridge forming residue D451 described above and that both residues are highly conserved in the consensus sequence of Ulp1-like molecules, suggesting that the N450S mutation in ulp1ts-333 is critical for Smt3 interaction and possibly substrate targeting. It is possible that N450S may interfere with the salt-bridge formed between D451 of Ulp1 and R64 of Smt3. In support of this hypothesis, correction of the N450S mutation in Ulp1(3)^{ts(S450N)} restores the ability of this truncation to interact with Smt3 in a two-hybrid assay (FIG. 5B) and partially rescues the growth defect a ulp1Δ strain (30° C.). Therefore, N450 and D451 appear to play a critical role in SUMO interaction.

In conclusion, we find that several features (N450, D451, and C580S) in region 3 of Ulp1, beyond the previously identified SBS domain, are required for targeting and retention at the septin ring.

### Example 4

### Kap121-Independent SUMO-Targeting Information Resides in Region 3 of Ulp1

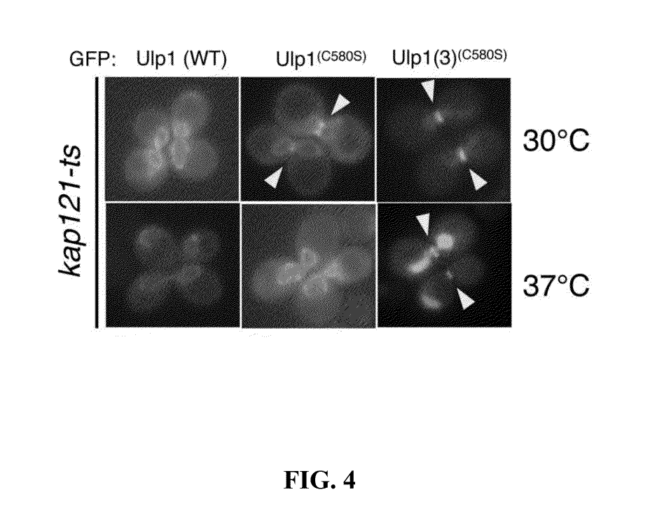

Region 3 of Ulp1 may not be the only domain involved in targeting to the septin ring. Region 1 of Ulp1, the Kap121-binding domain, has previously been implicated in septin-targeting. Specifically, Li et al. ((2003) Journal of Cell Biology 160, 1069-1081) reported that Kap121 is required for targeting Ulp1 to the septin ring during mitosis. Therefore, we decided to assess the role of Kap121 in the substrate-targeting of Ulp1(3^{)(C580S)}. Specifically, we used a kap121ts mutant to assess the septin ring-targeting of wildtype Ulp1, full-length Ulp^{1C580S}, and Ulp1(3^{)(C580S)}. Kap121ts cells were transformed with plasmids expressing GFP-tagged wildtype (WT) Ulp1, Ulp^{1(C580S)}, and Ulp1(3^{)(C580S)} under the control of the Ulp1 promoter (YOK 1487, YOK 1488, YOK 1944). Shown in FIG. 4 are representative images indicating the localization of GFP-tagged Ulp1 constructs in large-budded cells at 30° C. and 37° C., the non-permissive temperature for kap121-ts. The position of septin ring-localized Ulp1 constructs is indicated by arrowheads.

In our analysis, we found that full-length Ulp1^{C580S} required Kap121 function for targeting to the septin ring. At the permissive temperature (30° C.), Ulp1^{C580S} demarcated the nuclear envelope and septin ring of G2/M-arrested cells. After a shift to the non-permissive temperature, however, Ulp1^{C580S} could no longer be detected at the septin ring (FIG. 4, middle panel). Surprisingly, Ulp1(3)^{(C580S)} was localized to the septin ring at the permissive and non-permissive temperature in a kap121ts strain. As shown in FIG. 4 (right panel), Ulp1(3)^{(C580S)} resided both inside the nucleus and at the septin ring at 30° C. and 37° C.

Our data suggest that Ulp1 contains both Kap121-dependent and Kap121-independent septin ring targeting information. The only requirement to detect full- length Ulp1 at the septin ring is the C580S mutation and functional Kap121 (FIGS. 1, 2, and 4). In contrast Ulp1(3)^{(C580S)}, which lacks all domains required for NPC interaction through Kap121, Kap60, and Kap95, localizes to the septin ring and inside the nucleus. In summary, this finding provides strong evidence that Kap121-independent septin ring-targeting information resides in the catalytic domain (region 3) of Ulp1.

### Example 5

### Distinct and Separate Ulp1 Domains are Required for Interaction with SUMO

The finding that a single amino-acid change in the catalytic domain of Ulp1 yielded greatly enhanced, SUMO-dependent localization to the septin ring also prompted us to investigate the two-hybrid interactions of Ulp1 with budding yeast SUMO (Smt3-BD: Smt3 fused to the Gal4 DNA-binding domain). Referring to FIG. 5A, Ulp1 and Smt3 interactions were determined using yeast two-hybrid assays. The presence of both Smt3 (pOBD2/TRP1) and Ulp1 constructs (pOAD/LEU2) was confirmed by growth on medium lacking tryptophan and leucine (-T-L). The interaction between Ulp1 constructs and Smt3 is shown as duplicate spots of diluted cells on media lacking adenine (-A) (YOK 2163, YOK 2165, YOK 2167, YOK 2169, YOK 2171). See FIG. 3A for a graphic representation of individual constructs (Ulp1: full-length wildtype Ulp1; C580S: catalytically inactive; region 1: Ulp1(1-150) ; region 2: Ulp1(151-340); region 3: Ulp1(341-621)). FIG. 5B shows 2-hybrid analysis of mutated Ulp1 region 3 truncations with SUMO as indicated above (C580S: catalytically inactive; D451 N: deleted salt-bridge with SUMO; N450S: change in ulp1ts-333; S450N: N450S reverted to wildtype) (YOK 2173, YOK 2175, YOK 2177, YOK 2179, YOK 2181).

Full-length wildtype Ulp1, the full-length catalytically inactive Ulp1^{C580S} mutant, the Ulp1 Kap121-interacting domain (region 1), and the Ulp1 Kap60/Kap95-interacting domain (region 2), all failed to interact with Smt3-BD (FIG. 5A). However, both the catalytic domain (region 3) and the catalytically inactive Ulp1(3)^{(C580S)} mutant interacted with Smt3. The interaction of Ulp1(3)^{(C580S)} with Smt3 appeared much less pronounced than the wildtype Ulp1(3) interaction (4.5-fold reduced β-gal units) and could not be detected when diluted cells were spotted on media lacking adenine (FIG. 5B). It is likely that patching or spotting undiluted cells on media lacking adenine allowed us to detect the interaction between Ulp1(3)^{(C580S)} and Smt3 above background. Additional evidence suggesting that the Ulp1(3)^{(C580S)} mutant interacts avidly with Smt3 is provided below.

We focused on the important residues near the SBS domain (see FIG. 3B) of Ulp1 region 3. First, we investigated the D451 N mutant of Ulp1 that prevents the interaction of Ulp1 with SUMO (31,49). Indeed, when the D451 N mutation was introduced into region 3 of Ulp1, forming Ulp1 (3)^{(D451N)}, the interaction with Smt3 was abolished in our two-hybrid assay. The same mutation, when introduced into the full-length Ulp1^{(C580S)}, prevented localization to the septin ring (FIG. 3A). Second, region 3 of ulp1ts-333, Ulp1(3)^{ts}, failed to interact with Smt3. However, reverting a single mutation, N450S in Ulp1(3)^{ts}, back to the wildtype (N450) promptly restored the interaction with Smt3 (FIG. 5B-(3)^{ts} S450N). Therefore, we propose that many of the observed ulp1ts-333 phenotypes may be caused by defects in targeting and binding of critical sumoylated substrates in the cell.

The observation that the ts mutations in Ulp1(3)^{ts} weakened or disrupted the interactions with Smt3 suggests that these mutations could help explain the unexpectedly diminished levels of Smt3 interaction with the Ulp1(3)^{(C580S)} mutant. We reasoned that Ulp1(3)^{(C580S)}failed to score strongly with Smt3 because it was avidly interacting with free Smt3 or was sequestered by sumoylated proteins in the cell and, therefore, failed to interact with the BD-Smt3 fusion. Introduction of the ts mutations in Ulp1(3)^{ts} could weaken the substrate-trapping phenotype of Ulp1(3)^{(C580S)}, allowing it to regain the interaction with the BD-Smt3 fusion. Indeed, we found that combining these mutations in the Ulp1(3)^{ts (C580S)} construct reestablished the interaction with Smt3. This unique observation provides evidence that the targeting of Ulp1 to sumoylated substrates is a closely balanced act involving both Smt3 targeting and retention.

### Example 6

### The Ulp1(3)^{(C580S)} Truncation Binds SUMO and SUMO-Modified Proteins

We hypothesized that if Ulp1(3)^{(C580S)} were to interact avidly with Smt3, this mutated moiety of Ulp1 could efficiently interact with SUMO adducts in vitro. In order to test the direct interaction of Ulp1(3)^{(C580S)}with SUMO, we fused this domain to the carboxy-terminus of maltose-binding protein (MBP) and expressed the recombinant fusion protein in bacteria. Subsequently, the MBP-Ulp1(3)^{(C580S)} fusion protein was purified from bacterial extracts and bound to amylose resin. As a control to assess the ability of MBP-Ulp1(3)^{(C580S)} to interact with sumoylated proteins, we also purified a second MBP-fused Ulp1(3)^{(C580S)} construct lacking the SBS domain (3^{(C580S)}ΔSBS).

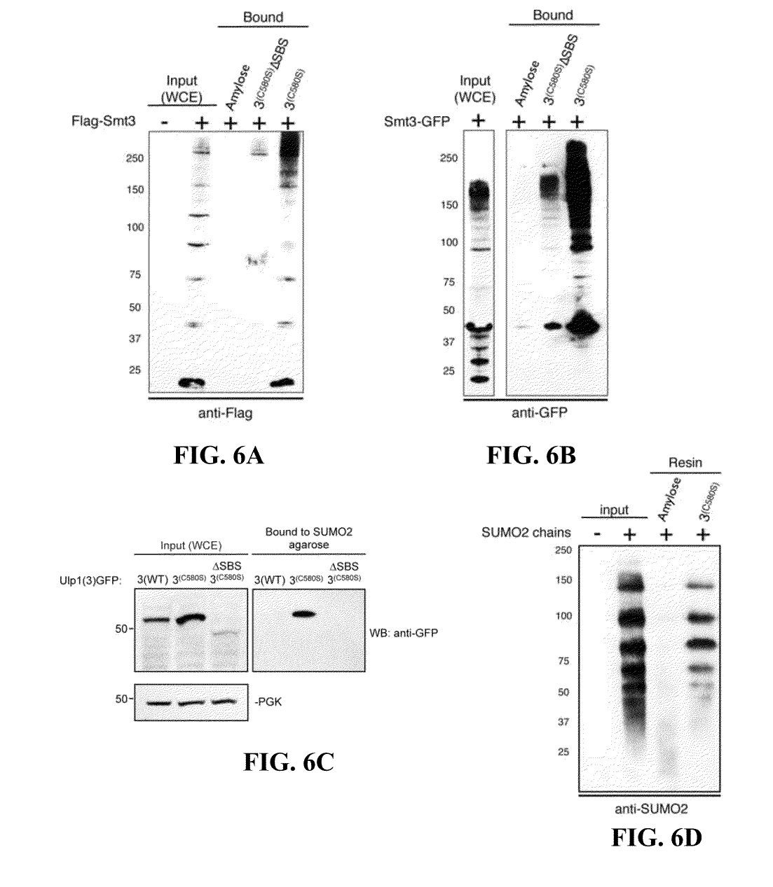

As shown in FIG. 6A and FIG. 6B, we demonstrated the ability of MBP-Ulp1(3)^{(C580S)} to affinity-purify sumoylated proteins from crude yeast cell extracts. Ulp1ts-333 cells expressing FLAG-tagged-SMT3 were grown to log-phase prior to preparation of yeast cell extracts. These extracts were then incubated with resin-bound MBP-Ulp1(3)^{(C580s)}, MBP-Ulp1(3)^{(C580S)}-ΔSBS, or unbound amylose resin. After washing, bound yeast proteins were eluted, separated on SDS-PAGE gels, and analyzed by Western blotting with an anti-FLAG antibody. Flag-SMT3-modified proteins present in the whole cell extracts (WCE) (FIG. 6A, lane 2) could clearly be detected bound to MBP-Ulp1(3)^{(C580S)} (FIG. 6A, lane 5) but not the MBP-Ulp1(3)^{(C580S)}-ΔSBS control protein (FIG. 6A, lane 4). We identified both unconjugated Flag-Smt3 proteins as well as several higher molecular weight adducts. These data suggest that Ulp1(3)^{(C580S)} can efficiently bind and enrich sumoylated proteins from crude yeast cell extracts. To demonstrate the versatility of Ulp1(3)^{(C580S)}-aided Smt3 purification, we also purified monomeric and conjugated GFP-Smt3 from yeast cells (FIG. 6B).

Additionally, we probed extracts and eluted proteins shown in FIG. 6B with an anti Cdc11 antibody, revealing the specific co-purification of of Cdc11 with immobilized Ulp1(3)^{(C580S)}. FIG. 6C shows SDS-PAGE images of immobilized Ulp1(3)^{(C580S)} that was used to affinity-purify Cdc11 from yeast WCEs. WCE containing GFP-Smt3 (YOK 1857) was prepared under non-denaturing conditions and incubated with immobilized MBP-Ulp1(3)^{(C580S)}(3^{(C580S)}),MBP-Ulp1(3)^{(C580S)} lacking the SUMO-binding surface (3^{(C580S)}ΔSBS) or unbound resin (amylose). After washing and elution, bound Cdc11 was detected using an anti-Cdc11 antibody (Santa Cruz Biotechnology). At right of FIG. 6C, WCEs from logarithmically growing yeast cells expressing GFP-tagged Ulp1(3), Ulp1(3)^{(C580S)}, Ulp1(3)^{(C580S)}ΔSBS (YOK 1839, YOK 1907, YOK 1903) (input) were prepared under non-denaturing conditions. Extracts were then incubated with SUMO2 immobilized on agarose beads (Boston Biochem). After washing and elution with sample buffer, bound proteins were detected using an anti-GFP antibody.

In the reciprocal experiment, we tested whether a GFP-tagged Ulp1(3)^{(C580S)} construct expressed in yeast cells could bind immobilized SUMO2, which is highly conserved to yeast Smt3. In this experiment, yeast cells expressing CEN-plasmid levels of the GFP-tagged Ulp1(3), Ulp1(3)^{(C580S)}, or the Ulp1(3)^{(C580S)}-ΔSBS (see FIG. 3) were grown to log-phase prior to preparation of yeast cell extracts. Individual extracts were then incubated with SUMO2 immobilized on agarose beads as described above. After washing, bound yeast proteins were eluted, separated on SDS-PAGE gels, and analyzed by Western blotting with an anti-GFP antibody. This time, the GFP-tagged Ulp1(3)^{(C580S)} could be detected in the WCE and bound to the SUMO2 agarose (FIG. 6C at right). In contrast, neither the wildtype catalytic domain of Ulp1 (Ulp1(3)) nor Ulp1(3)^{(C580S)}(SBSΔ) bound to SUMO2-agarose. Similarly, the Ulp1(3)^{(C580S)} could also be purified on SUMO-1 agarose.

We also analyzed if immobilized Ulp1(3)^{(C580S)} could be used to purify SUMO chains. For this experiment, we incubated purified SUMO2 chains with our immobilized Ulp1(3)^{(C580S)} or the unbound amylose resin. After washing, bound SUMO2 chains were eluted, separated on SDS-PAGE gels, and analyzed by Western blotting with an anti-SUMO2 antibody. SUMO2 chains could clearly be detected in the input (FIG. 6D—lane 2) and bound to the MBP-Ulp1(3)^{(C580S)} (lane 4), but not to the resin-only control (FIG. 6D—lane 3). Both lower and higher molecular weight adducts of SUMO2 were purified with preference for higher molecular weight chains (5-7 mers). These data suggest that the Ulp1(3)^{(C580S)} can efficiently bind and enrich SUMO2 chains in vitro and that the MBP fusion of Ulp1(3)^{(C580S)} may also be useful for the purification of sumoylated proteins from mammalian cells.

### Example 7

### MBP-Ulp1(3)^{(C580S)} can Serve as a SUMO2 Binding Platform for STUbL-Mediated Substrate Ubiquitylation

SUMO-targeted ubiquitin ligase proteins (STUbLs) (e.g., the yeast Slx5/Slx8 heterodimer and the human RNF4 protein) efficiently ubiquitylate proteins modified with SUMO chains (51,52). These proteins interact with their respective sumoylated ubiquitylation targets through SIMs. STUbL reactions have been reconstituted in vitro, but the purification of target proteins modified with SUMO chains has been technically difficult and/or prohibitively expensive. The ability of Ulp1(3)^{(C580S)} to interact with SUMO can provide a simple way to purify a SUMO-chain-modified STUbL target of choice.

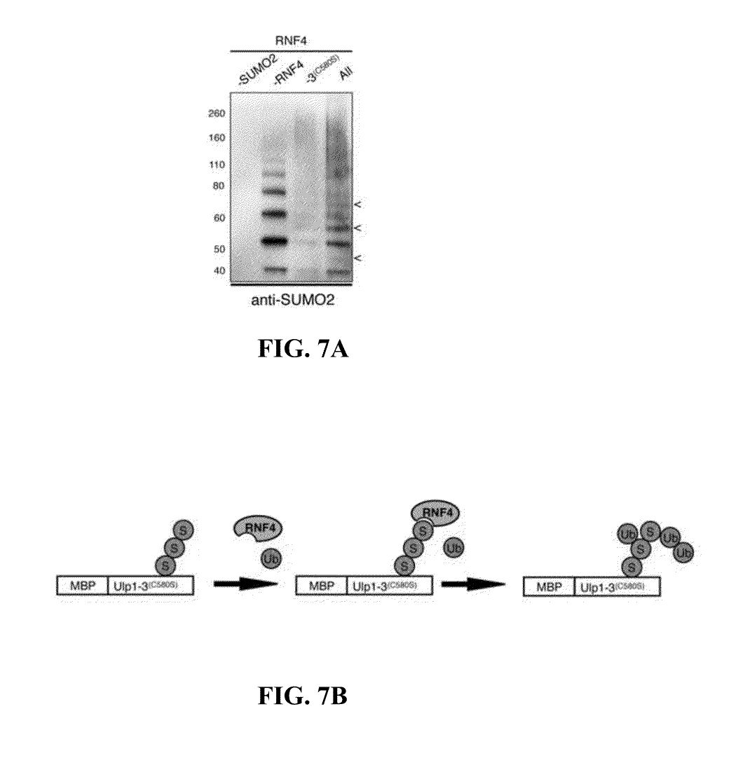

To demonstrate that Ulp1(3)^{(C580S)} can serve as a platform to modify a purified protein with SUMO2 chains, we incubated the immobilized MBP-Ulp1(3)^{(C580S)} with SUMO2 chains. Unbound SUMO2 chains were removed by washing. The MBP-Ulp1(3)^{(C580S)} SUMO2 chain complex was then eluted and added into a STUbL in vitro ubiquitylation reaction containing recombinant RNF4 (Fryrear and Kerscher, unpublished reagents). Proteins in the STUbL-mediated ubiquitylation assay were separated on SDS-PAGE gels and analyzed by Western blotting with an anti-SUMO antibody. Referring to FIG. 7A, arrows indicate modified SUMO2 chains (lane 1: no SUMO chains; lane 2: no RNF4; lane 3: no Ulp1(3)^{(C580S)}; lane 4: all reagents). Consistent with previous observations, we were able to detect ubiquitylated SUMO2 chains after the STUbL reaction. This ubiquitylation was dependent on RNF4 and SUMO2 chains. Based on these results, we propose that the Ulp1(3)^{(C580S)} may provide a useful, widely applicable tool for the study of sumoylated proteins and STUbL targets. FIG. 7B depicts a schematic model for using MBP-Ulp1(3)^{(C580S)} as a SUMO2 binding platform for substrate ubiquitylation. SUMO2, ubiquitin, and RNF4 are indicated by spheres labeled S, spheres labeled Ub, and the gray oval labeled RNF4, respectively.

### Example 8

### Purification and Mass Spectrometry-Aided Identification of Sumoylated Proteins from Complex Mixtures of Yeast Proteins

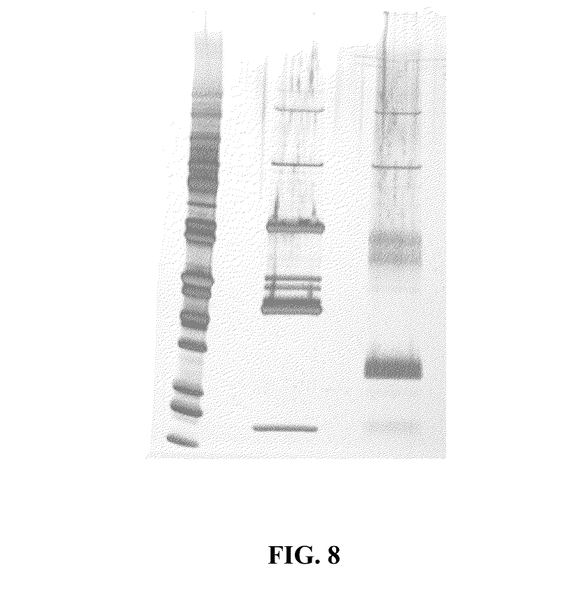

YOK428, aulp1::KANmx deletion strain, carries plasmids expressing both temperature sensitive ulp1ts under control of the native ULP1 promoter as well as FLAG-tagged conjugation competent SUMO (SUMOgg-FLAG) under control of the yeast GPD promoter. Two liters of YOK428 cells were grown to late log-phase in media containing G418 and lacking leucine. Cells were then harvested by centrifugation at 4° C. and pellets were washed once with 1 L ice cold wash buffer (50 mM HEPES, 3 mM DTT, 2% dextrose). Cells were then re-suspended in a volume of extrusion buffer (50 mM HEPES 7.8, 325 mM NaCl, 14 mM BME, 5 mM MgCl₂, protease inhibitors) equal to that of the packed cell volume. Cells were spun down and most of the supernatant was discarded. The remaining cell paste was scraped into a 10 mL syringe and snap-frozen by extruding the paste into a 50 mL centrifuge tube containing liquid nitrogen, resulting in high cell density yeast noodles. Cells were lysed in a cryogenic tissue grinder, and the resulting powder was placed at −80° C. to allow the dry ice to sublimate. Powdered yeast lysate was dissolved in 10 ml extraction buffer (50 mM HEPES 7.8, 325 mM NaCl, 14 mM BME, 5 mM MgCl₂, 10% glycerol, protease inhibitors·) and snap frozen in liquid nitrogen. Protein extracts were thawed on ice and insoluble cellular components were spun out of solution. 10 mM NEM was added to inhibit desumoylating enzymes and a 30 μl aliquot was taken as a whole cell extract control. The remaining volume was gravity-fed through a 1 ml bed of either washed, equilibrated resin-bound MBP-Ulp1(3)^{(C580S)} (“U-tag resin”) or an amylose control resin. The U-Tag and amylose resins were washed twice with 5 bed volumes of column wash buffer (50 mM HEPES, 325 mM NaCl, 1% Triton x-100) each. The beads were then removed to 1.7 mL eppendorf tubes and sumoylated proteins were eluted by processing with recombinant Ulp1 protease. Specifically, SUMO bound resin was incubated with 10U of SUMO-HIS₆ₓ protease for 6 hours in 1× SUMO protease buffer with salt. After processing, beads were collected by brief centrifugation at <800 xg and the supernatant containing SUMO substrates was put through a PrepEase (USB corporation) His affinity column to remove the HIS₆ₓ tagged Ulp1 SUMO protease. Samples of whole cell extracts and eluted proteins were analyzed by SDS-PAGE as shown in FIG. 8. Approximately 50 ng of total protein was loaded onto a precast NuPAGE 4-12% Bis-Tris gel (Invitrogen) and electrophoresed at 200 v for 50 minutes. The gel was then silver stained using a Pierce Silver Stain for Mass Spectrometry kit (Thermo Scientific) as per the manufacturer's instructions. In comparison to mock-purified samples (see FIG. 8, third lane) from the amylose resin, 33 distinct proteins (9 present with 3 or more fragments), eluted from the U-tag resin (see FIG. 8, middle lane). One of the purifying proteins was identfied as yeast SUMO (Smt3) and several others have previously been shown to be modified with SUMO.

### Discussion of Examples 1-8

Region 3 of Ulp1, the catalytic domain, contains critical information for the subcellular targeting to sumoylated substrates, including the septin Cdc11. To determine how Ulp1 is targeted to its substrates, we took advantage of a catalytically inactive Ulp1 mutant (C580S) that exhibited a partial redistribution from the nuclear envelope to the septin ring of dividing yeast cells. The re-localization of Ulp1 depended on functional Smt3 and sumoylated proteins at the septin ring of dividing cells.

Using this novel Ulp1 in vivo septin-ring localization assay, we traced the critical targeting information to two features in region 3 of Ulp1, a previously identified SUMO-binding surface (SBS) (amino acids 418-447) and two residues (N450 and D451) that reside near the carboxy-terminus of Smt3. D451 of Ulp1 contacts Smt3 through a salt bridge interaction. In contrast, N450, a residue that is mutated in the ulp1ts allele (N450S), does not seem to contact Smt3. Therefore, it is possible that perturbation of the D451 salt-bridge, due to the juxtaposed N450S mutation, results in the reduced ability to dock Smt3 in place once it has contacted the SBS domain.

The sole requirement for the enrichment of full-length Ulp1 at the septin ring was the catalytically inactivating C580S mutation in the catalytic domain of Ulp1. This finding has important implications for the targeting role played by the amino-terminal karyopherin binding domains of Ulp1. Additionally, catalysis of Smt3 appears to be required for substrate release. The catalytically inactive Ulp1(3)^{(C580S)} mutant is predominantly localized to the septin ring and nucleus of dividing yeast cells, while the catalytically active wildtype Ulp1(3) shows merely a diffuse staining throughout the cell. The C580S mutation may trap a bound Smt3 protein, allowing it to be observed in association with cellular desumoylation substrates. In support of this assessment, combining the D451 N with the C580S mutation abolished all visible bud-neck localization (FIG. 3A).

The interaction of budding yeast Ulp1 with Smt3 relies on multiple hydrophobic and salt bridge interactions between the catalytic domain (region 3) of Ulp1 and the carboxy-terminal extension of Smt3.

Our research demonstrates for the first time that non-covalent interactions between Ulp1 and SUMO are not only important for SUMO binding, but also for the cytosolic targeting of this SUMO protease to the bud-neck and potentially sumoylated septins. Sumoylated proteins that accumulate or aggregate in the cytosol of yeast cells may be readily detectable by Ulp1(3)^{(C580S)}. Ulp1(3)^{(C580S)} also provides a useful tool to purify these sumoylated proteins. In conclusion, our findings provide strong evidence that SUMO, at least in the case of sumoylated proteins at the septin ring, is a required signal for the cytoplasmic targeting of Ulp1.

One intriguing aspect of the above Examples is the analysis of the substrate-trapping Ulp1(3)^{(C580S)} construct. Three lines of evidence reveal the avid interaction of Ulp1(3)^{(C580S)}with SUMO proteins and sumoylated substrates. First, this Ulp1-derived construct shows a pronounced interaction with the bud-neck comprised of sumoylated septins in vivo. Second, the reduced interaction of Ulp1(3)^{(C580S)}with Smt3 in a two-hybrid assay can be re-established by the introduction of mutations that weaken the interaction with Smt3. Third, the purified, recombinant Ulp1(3)^{(C580S)} protein is a potent affinity-tag for the purification of Smt3 conjugates and SUMO-modified proteins. A related study involving the C603S mutant of the human SENP1 protease confirms our assessment of the substrate-trapping feature. The authors observe re-localization of their SENP1(C603S) mutant in vivo to PML nuclear bodies and domains of the HDAC4 protein, suggesting that SUMO-dependent-targeting may be a conserved feature of Ulp1-like SUMO proteases. The latter may also provide a useful strategy for the identification of mitotically important desumoylation substrates. For example, two-hybrid screens with Ulp1(3)^{(C580S)} have already identified several novel cytosolic desumoylation targets.

### INCORPORATED BY REFERENCE

All publications, patents, and patent applications cited herein are hereby expressly incorporated by reference in their entirety and for all purposes to the same extent as if each was so individually denoted.

Equivalents

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

The articles “a” and “an” are used herein to refer to one or to more than one (i.e. to at least one) of the grammatical object of the article. By way of example, a protein means one protein or more than one protein.

Any ranges cited herein are inclusive.

We claim:

1. An isolated polypeptide comprising amino acids 1 through 219 of SEQ ID NO: 1.

2. A Ulp1 polypeptide variant that binds to SUMO comprising a polypeptide having at least 80% identity with the amino acid sequence of SEQ ID NO: 1.

3. A Ulp1 polypeptide variant of claim 2 comprising an amino acid sequence with 1 to 10 residue modifications relative to the amino acid sequence shown in SEQ ID NO: 1, wherein amino acid residue 178 of said Ulp1 polypeptide variant comprises serine.

4. A Ulp1 polypeptide variant of claim 3 additionally comprising a covalently bonded fluorescent tag.

5. A DNA isolate encoding a polypeptide comprising at least 80% identity with the amino acid sequence of SEQ ID NO: 1.

6. A DNA isolate of claim 5 wherein said DNA isolate encodes SEQ ID NO: 1.

7. A recombinant expression vector comprising the DNA isolate of claim 6.

8. A fusion protein comprising Ulp1(3)^{(C580S)}.

9. A fusion protein of claim 8 comprising a Ulp1(3)^{(C580S)} protein fused at the C terminus with a protein or peptide capable of being fused to Ulp1(3)^{(C580S)}.

10. A fusion protein of claim 8 comprising a Ulp1(3)^{(C580S)} protein fused at the N terminus with a protein or peptide capable of being fused to Ulp1(3)^{(C580S)}.

11. A method for binding a SUMO-tagged protein comprising mixing a SUMO-tagged protein with a Ulp1 polypeptide variant comprising an amino acid sequence with 0 to 10 residue modifications relative to the amino acid sequence shown in SEQ ID NO: 1, wherein amino acid residue 178 of said Ulp1 polypeptide variant comprises serine.

12. A method of claim 11 wherein said polypeptide comprises amino acids 1 through 219 of SEQ ID NO: 1.

13. A method of claim 11 wherein said Ulp1 polypeptide variant is a fluorescent-labeled Ulp1 polypeptide variant.
